(19) United States
(12) Patent Application Publication
(10) Pub. No.: US 2002/0062830 A1
(43) Pub. Date: May 30, 2002

(54) Respirator for a protective device, such as a protective mask, protective hood or protective suit

(75) Inventors: Peter Meier, Seuzach (CH); Christian Suter, Wettingen (CH); Daniel Giger, Holziken (CH)

Correspondence Address: WEBB ZIESENHEIM LOGSDON ORKIN & HANSON, P.C., William H. Logsdon, 700 Koppers Building, 436 Seventh Avenue, Pittsburg, PA, 15219-1818, US

(73) Assignee: Micronel AG, Tagelswangen (CH)

(21) Appl. No.: 09/975,770

(22) Filed: Oct. 11, 2001

(30) Foreign Application Priority Data
Oct. 11, 2000 (CH) 2003/00

(51) Int. Cl. A62B 7/00; A61M 16/00

(52) U.S. Cl. 128/206.12; 128/204.21

(57) ABSTRACT

A respirator has a filter that is located in an air duct. A blower has an air conduction housing and an impeller wheel located in said air conduction housing, with an impeller wheel that is rotationally mounted inside said housing. The blower is controlled by a sensor. The output of the blower is controlled by means of an electronic control circuit. The sensor is a volume flow or mass flow sensor located in the air duct, and is independent of the blower and controls the blower by means of the control circuit so that the flow of respiratory air remains essentially constant.

### BACKGROUND OF THE INVENTION

1. Field of the Invention

This invention relates to a respirator for a protective device such as a protective mask, protective hood or protective suit, for example, with a filter located in an air duct, a fan that has an air conduction housing and an impeller wheel located in said air conduction housing. The fan is controlled by a sensor, with a motor for the drive of the impeller wheel, as well as an energy source and an electronic control circuit that controls the output of the fan.

2. Brief Description of the Prior Art

Respirators of this type are well known. The fan generates an air current and thereby assists respiration. The air current that is fed to the protective device should be kept as constant as possible and should correspond to a setpoint. One problem with protective devices of the prior art is that the filter located in the air duct becomes clogged during use. The resistance therefore increases and the air flow decreases. An additional problem is that different filters have different resistances, and the air flow is a function of the filter that is installed.

German Patent 195 06 360 describes a respirator in which the output of the blower is regulated on the basis of the current and the speed of rotation of the fan. The quantity of the airflow fed to a gas mask can thereby be kept constant. In this case, the blower acts as a detector, by means of which its output is regulated. For this purpose, capacitive electrodes are located on the blower which transmit information corresponding to the speed of the impeller wheel by means of an oscillator and a phase-locking loop to a meter of a microcontroller. The current of the blower motor is measured and transmitted via a current-measuring amplifier to the A/D converter of the microcontroller. The capacitive electrodes are connected with the impeller of the fan in a circuit, whereby changes in the capacity of these electrodes result in a variation in the frequency of the oscillator. The air current should therefore be independent of the filter resistance. However, the electronic system required is relatively expensive and complicated.

### SUMMARY OF THE INVENTION

The object of the invention is to create a respirator of the type described above that is easier and more economical to manufacture, and that nevertheless is essentially insensitive to any interference or variations that may be caused by contamination of the filters.

The invention teaches that the sensor is a volume or mass flow sensor located in the air duct, which sensor is independent of the blower and which controls the blower via the control circuit so that the current of respiration air remains essentially constant. The respirator according to the invention makes possible a closed-loop control system that is independent of the air resistance of the system and of the ambient temperature. It has been shown that the use of a sensor that is independent of the blower makes possible a very precise regulation of the flow of respiration air. Filters that have different air resistances each result in the same flow of respiration air. As a result of the precise closed-loop control system, the current consumption can be reduced and the battery life can be extended, among other advantages.

One development of the invention results in a particularly economical and reliable realization of the invention if the sensor is embodied in the form of a fan with a freely rotating impeller wheel and has an apparatus for the sensing element. The sensing element device can be a Hall-effect element, a photoelectric barrier or an inductive or capacitive sensor. Sensing elements of this type are economical elements and result in an accurate sensing signal. A suitable electronic interface converts the signal from the sensor into a control signal for the blower. The controlled variable is preferably an air flow setpoint.

Additional advantageous features of the invention are disclosed in the claims, in the following description and in the accompanying drawings.

### BRIEF DESCRIPTION OF THE DRAWINGS

One exemplary embodiment of the invention is explained in greater detail below and is illustrated in the accompanying drawings, in which:

FIG. 1 is a schematic illustration of a respirator according to the invention with a gas mask, which is not shown here in any further detail, as the protective device,

FIG. 2 is a schematic illustration of a regulated closed-loop control circuit,

FIG. 3 is a schematic view of a sensor in the form of a fan,

FIG. 4 is an additional schematic view of the sensor illustrated in FIG. 3,

FIG. 5 is a diagram with measurement curves of a closed-loop flow control system,

FIG. 6 is a diagram like the one shown in FIG. 1, whereby the current limitation is 420 mA.

### DETAILED DESCRIPTION OF THE INVENTION

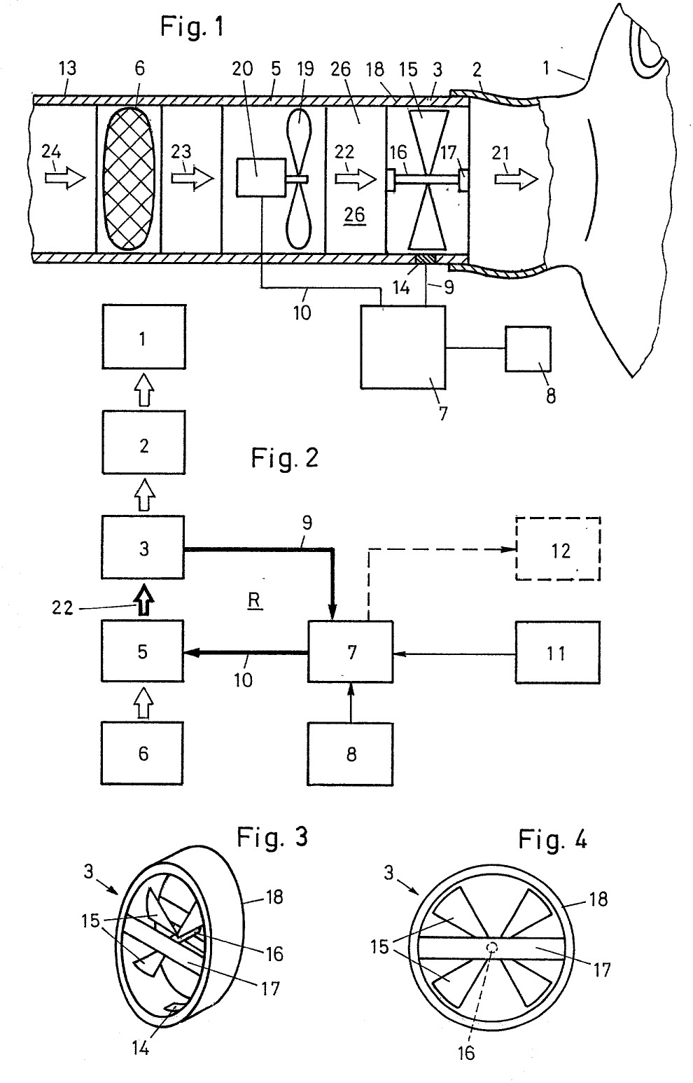

The respirator illustrated in FIG. 1 has an air hose 2 which is connected so that it feeds air in the direction indicated by the arrow 21 to a protective mask 1 or to another protective device, such as a protective hood or a protective suit, for example. The air fed to the protective mask 1 is sucked in at a nozzle 13, for example, and fed in the direction indicated by the arrow 24 to at least one filter 6. The filter 6, which is here shown only schematically, can be a carbon filter, for example, or any other suitable filter. A plurality of filters 6 can also be provided. The air cleaned in the filter 6 travels in the direction indicated by the arrow 23 to a blower 5 which is preferably a radial blower and in the conventional manner has an impeller wheel 19 which is driven by an electric motor 20. The electric motor 20 is fed from an energy source 8, such as a battery for example, a rechargeable storage battery or from an external power source.

The blower 5 transports the air in the direction indicated by the arrow 22 in an air duct 26 to an air flow sensor 3 which, like the blower 5, has an air conduction housing 18 and an impeller wheel 15 rotationally mounted in the air conduction housing 18. The impeller wheel 15 is not driven, however, but rotates passively on account of the current of air that passes through the air conduction housing 18 to the hose 2. The mass of the impeller wheel 15 is kept as small as possible, and the bearing 16 preferably has the lowest possible resistance. The speed of rotation of the impeller wheel 15 is proportional to the volume or mass flow that passes through the air conduction housing 18.

FIGS. 3 and 4 illustrate the construction of the air flow sensor 3. As shown, the air conduction housing is cylindrical and has webs 17 at intervals from one another that run radially, on which the bearing 16 is fastened. A primary detector 14 is located on the air conduction housing 18 so that it responds to rotations of the impeller wheel 15. The primary detector 14 is preferably a Hall-effect element, although inductive or capacitive primary detectors could also be used. Ultimately, other types of detector elements, such as optical detectors, are also possible. Detector elements of this type are themselves disclosed in the prior art and are commercially available.

As the impeller wheel 15 turns, the primary detector 14 generates a signal that is proportional to the speed of rotation and is transmitted via a communications line 9 to a control circuit 7. This control circuit 7 is connected by means of an additional line 10 to the motor 20 of the blower 5 and regulates the motor so that the current of air delivered by the respirator remains essentially constant. As shown, the air flow sensor is independent of the blower 5.

FIG. 2 shows, among other things, the closed-loop control circuit R which is formed by the air flow sensor 3, the control circuit 7 and the blower 5, the lines 9 and 10 and the air current 22. The air current 22 runs through the air duct 26 and the air conduction housing 3 and ultimately travels through the air hose 2 into the protective mask 1. A setpoint for the air flow can be fed to the control circuit 7 by means of a switching element 11.

The operation of the respirator according to the invention is describedhereinafter.

A desired air flow setpoint, such as 120 l/min, for example, is set on the switching element 11. If the respirator is then turned on by means of a switch (not shown here), the impeller wheel 19 of the blower begins to rotate and generates an air flow 22 which is directed in the air duct 26 in the direction indicated by the arrow 22 toward the air flow sensor 3. Excited by this air flow 22, the impeller 15 rotates, whereupon the primary detector 14 is excited corresponding to the speed of rotation and produces a corresponding signal. On the basis of this signal, the control circuit 7 controls the control system of the motor 20 until the air flow setpoint is reached. After this setpoint is reached, the air flow is kept constant by the closed-loop control circuit R, whereby the electronic control system, in a manner described by the prior art, compares a setpoint signal with a measured signal and thus establishes an equilibrium.

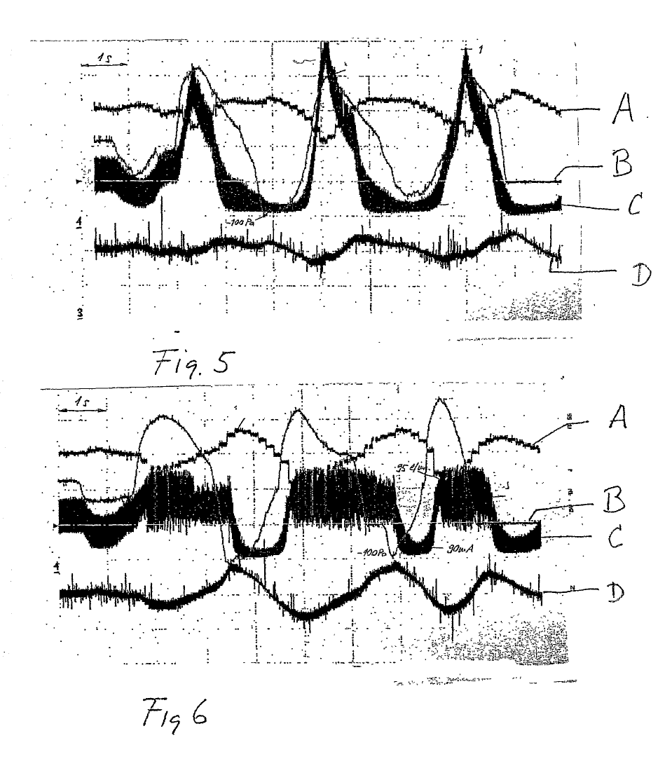

When the user breathes through the protective mask 1, the above mentioned equilibrium is upset, so that as shown in FIG. 5, the air current A, the air pressure B and the blower current C change. The closed-loop control circuit R works as explained above to maintain the equilibrium. To conserve energy, there is preferably a current limiting device. The measurement illustrated in FIG. 5 is performed without current limiting, while the measurement illustrated in FIG. 6 is performed with a current limitation of 420 mA. The control signal from the sensor is indicated by the curve D in these two FIGS. 5 and 6. The control signal D from the sensor 3 runs essentially parallel to the air flow curve A. The curve of the air pressure B, on the other hand, runs essentially diametrically opposite to the curve of the air flow A.

If the air resistance then changes, for example as a result of a contamination of the filter 6, the impeller wheel 15 rotates more slowly, because the blower 5 is transporting less air. The closed-loop control device 7 then executes the corresponding regulation until the setpoint for the air flow 22 is achieved again. The same thing happens when a new filter 6 with a different air resistance is used. When a filter that has a higher air resistance is used, the blower 5 is regulated so that its output is greater, corresponding to the higher resistance.

The control circuit 7 can have an indicator 12 which can indicate the battery status or the air flow set point, for example. The indicator can be optical, acoustical or even vibrating.

Having described presently preferred embodiments of the invention, it is to be understood that it may be otherwise embodied within the scope of the appended claims.

We claim:

1. A respirator for a protective device, such as a protective mask, protective hood or protective suit, comprising at least one filter located in an air duct, a blower having an air conduction housing and an impeller wheel located in it, a motor for the drive of the impeller wheel, which impeller wheel is controlled by a sensor, an energy source and an electronic control circuit, which controls the output of the blower, wherein the sensor is a volume flow or mass flow sensor located in the air duct, which sensor is independent of the blower and controls the blower by means of the control circuit so that the flow of respiratory air remains essentially constant.

2. The respirator as claimed in claim 1, wherein the sensor has a free-rotating impeller wheel which is driven by the air flow.

3. The respirator as claimed in claim 2, wherein the impeller wheel works together with a primary detector.

4. The respirator as claimed in claim 2, wherein the impeller wheel is mounted in a cylindrical air conduction housing.

5. The respirator as claimed in claim 2, wherein the sensor has a primary detector on a housing.

6. The respirator as claimed in claim 5, wherein the primary detector is a Hall-effect element or an optical primary detector.

7. The respirator as claimed in claim 5, wherein the primary detector is an inductive or capacitive primary detector.

8. The respirator as claimed in claim 1, wherein the sensor, viewed in the direction of the flow, is located downstream of the blower.

9. The respirator as claimed in claim 1, wherein the sensor, viewed in the direction of the flow, is located upstream of the blower.

10. The respirator as claimed in claim 1, wherein the blower is a radial blower.
